(19) United States
(12) Patent Application Publication
(10) Pub. No.: US 2005/0228328 A1
(43) Pub. Date: Oct. 13, 2005

(54) Burn, sunburn, and cellulite treatment system

(76) Inventor: Linda A. Carter, Irving, TX (US)

Correspondence Address: Michael A. O'Neil;Michael A. O'Neil, P.C., Suite 820, 5949 Sherry Lane, Dallas, TX, 75225, US

(21) Appl. No.: 11/146,805

(22) Filed: Jun. 7, 2005

Related U.S. Application Data
(63) Continuation-in-part of application No. 10/642,915, filed on Aug. 18, 2003.
(60) Provisional application No. 60/404,444, filed on Aug. 19, 2002.

(51) Int. Cl. A61F 13/00

(52) U.S. Cl. 602/44; 26/71

(57) ABSTRACT

A treatment fluid dispensing apparatus comprises a fluid impervious layer and an overlying treatment fluid dispensing layer. The fluid treatment dispensing layer comprises a finely woven fabric formed from natural or synthetic fibers which normally retains the treatment fluid and which dispensing the treatment fluid responsive to pressure and/or body temperature. The treatment fluid dispensing layer is configured to dispense treatment fluid to areas of the human body over a prolonged period of time.

### CROSS-REFERENCE TO RELATED APPLICATIONS

This application is a continuation-in-part of application Ser. No. 10/642,915 filed Aug. 18, 2003, currently pending, which claims priority of provisional Application Ser. No. 60/404,444 filed Aug. 19, 2002.

### TECHNICAL FIELD

This invention relates generally to the application of fluids to the human body, and more particularly to the application of burn, sunburn, and cellulite treatment fluids to the entire body or portions thereof over an extended period of time.

### BACKGROUND AND SUMMARY OF THE INVENTION

As is well known, burn and sunburn damage to the skin is often treated by the application of treatment fluids. As used herein, the term fluid includes liquids, gels, lotions, creams, and all other non-solid flowable materials. The term treatment fluids includes over-the-counter preparations and also includes preparations prescribed by doctors for the treatment of more serious burn or sunburn damage.

Treatment fluids almost always require continuous application in order to achieve maximum benefit. However, the daily activities of most people preclude the continuous application of treatment fluids which results either in ineffective treatment or the requirement for frequent re-application of the treatment fluid being used.

The present invention relates to the continuous application of treatment fluids to all or portions of the human body which overcomes the foregoing and other problems which have long since characterized the prior art. In accordance with the broader aspects of the invention, there is provided a two layer structure, including a first fluid impervious layer which retains a treatment fluid and a second layer formed from a fine mesh of either natural or synthetic fibers which normally retains the treatment fluid but which releases the treatment fluid upon the application of pressure and/or body temperature. The treatment fluid dispensing layer may be subdivided in accordance with predetermined patterns better to control the release of treatment fluid therethrough.

In accordance with the present invention, there is provided a full body treatment suit adapted to cover the entire torso, arms, and legs of a person being treated for burn, sunburn related skin damage, or cellulite treatment. Depending on the nature of the injury, condition, or problem being treated, selected portions of the full body treatment suit can be removed by cutting. Alternatively, the full body treatment suit may comprise a plurality of sections which are selectively joined together by conventional fasteners such as hook-and-loop type fasteners, snaps, buttons, zippers, etc. The full body treatment suit may comprise a fluid dispensing layer comprising a plurality of predetermined patterns which are aligned with particular sections of the body.

### BRIEF DESCRIPTION OF THE DRAWINGS

A more complete understanding of the present invention may be had by reference to the following Detailed Description when taken in connection with the accompanying Drawings, wherein:

FIG. 1 is a diagrammatic illustration of the invention.

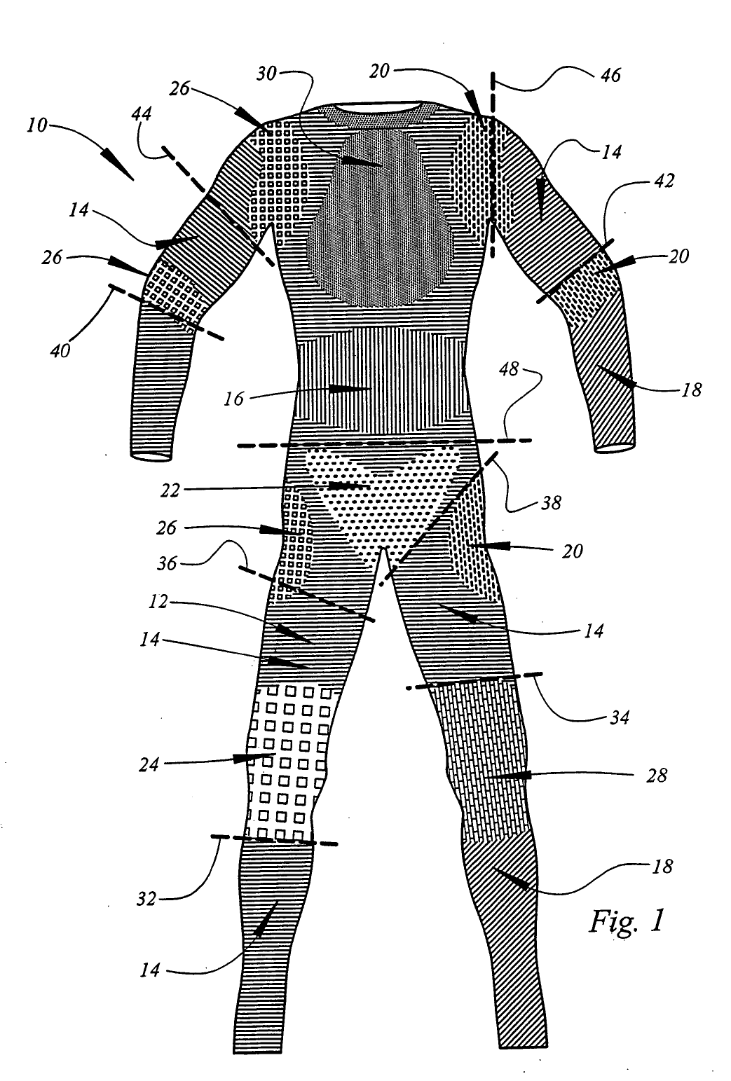

### DETAILED DESCRIPTION

Referring now to the Drawing, there is shown an apparatus for applying treatment fluids to the human body 10 comprising the present invention. The apparatus 10 comprises a full body suit or covering which covers the torso, arms, and legs of a person receiving treatment. The apparatus 10 includes a fluid impervious layer (not shown) and a skin engaging fluid dispensing layer 12. The layer 12 is formed from finely woven natural or synthetic fibers, and is configured to normally retain a treatment fluid therein and to cause treatment fluid to flow therethrough upon the application of pressure and/or body temperature.

The treatment fluid dispensing layer 12 may assume a wide variety of configurations. For example, the layer 12 may comprise spaced, parallel transversely extending sections 14; or spaced, parallel longitudinally extending sections 16; or spaced, parallel angularly extending sections 18. Portions of the treatment fluid dispensing layer 12 may comprise a generally open configuration which is secured to the overlying fluid impervious layer by a plurality of connection points as shown at 20 and 22. The treatment fluid dispensing layer 12 may also comprise large individual sections 24 or small individual sections 26 configured to dispense smaller amounts of treatment fluid. Another option is to configure the treatment fluid dispensing layer 12 in a checkerboard pattern 28 which affords precise control over the dispensing of treatment through the treatment fluid dispensing layer 12. The treatment fluid dispenser layer may also comprise a very fine mesh 30. As will be appreciated by those skilled in the art, the treatment fluid dispensing layer 12 may be configured in a wide variety of configurations depending upon the requirements of particular applications of the invention.

In accordance with one embodiment of the invention, the treatment fluid dispensing apparatus 10 is supplied in a full body configuration which covers the torso, legs, and arms of the person being treated. Depending upon the requirements of particular applications of the invention, the suit or covering comprising the apparatus 10 may be cut below the knee as indicated by the line 32, above the knee as indicated by the line 34, at mid-thigh as indicated by the line 36, at the intersection of the leg and torso as indicated by the line 38, below the elbow as indicated by the line 40, above the elbow as indicated by the line 42, below the shoulder as indicated by the line 44, or at the shoulder as indicated by the line 46. The suit or covering comprising the apparatus 10 may also be separated at the waist as indicated by the line 48 thereby providing upper and lower body components.

In accordance with another embodiment of the invention, the suit or covering comprising the apparatus 10 is supplied in a plurality of components, such as lower leg components, an upper leg/lower torso component, an upper torso component, and arm components. If so supplied, the components comprising the apparatus 10 are adapted for connection by conventional fasteners such as hook-and-loop type fasteners, snaps, buttons, zippers, etc.

In accordance with yet another embodiment of the invention, the suit or covering comprising the apparatus 10 is supplied in a plurality of components, such as strips having various widths and lengths, patches comprising a variety of sizes, disks comprising a variety of diameters, etc. If so supplied, the components comprising the apparatus 10 are adapted for connection by conventional means such as adhesives of the type utilized in the construction of bandages. The strips, patches, and disks may also be secured utilizing adhesive tape and/or gauze in the conventional manner.

It will therefore be understood that the present invention comprises a structure which extends across the entire body of an individual or one or more selected portions thereof to effect the application of treatment fluids thereto. A treatment fluid dispensing layer normally retains the treatment fluid to be applied, but responds to the application of pressure and/or body temperature to dispense the treatment fluid in controlled amounts. In this manner the treatment fluid is applied over an extended period of time.

The suit or covering comprising the apparatus 10 is designed in such manner so that an individual can wear the lightweight apparatus comfortably under his/her clothing. Thus, a person with a sun burn can be treated with sun burn medication dispensed through the treatment fluid dispensing layer 12 without staining his/her clothing. This apparatus also has a beneficial use of minimizing the appearance of cellulite on an individual's leg, arm, buttocks, etc.

In the case of severely burned patients, the present invention can also take the form of a sheet which is laid over the burned portions of the patient's body. The invention can also take the form of poncho-like construction having a hole for receiving the head of the patient and thereafter engaging the chest, back, and arms of the patient. The invention can also take the form of a mask-like covering for the face of the patient.

Although preferred embodiments of the invention have been illustrated in the accompanying Drawings and described in the foregoing Detailed Description, it will be understood that the invention is not limited to the embodiments disclosed, but is capable of numerous rearrangements, modifications, and substitutions of parts and elements without departing from the spirit of the invention.

1. An apparatus for applying treatment fluids to the human body comprising:
a first fluid impervious layer; and
a treatment fluid dispensing layer secured to and overlying the fluid impervious layer;
the treatment fluid dispensing layer comprising a finely woven fabric which normally retains the treatment fluid and which dispenses the treatment fluid in response to pressure and/or body temperature; and
the first fluid impervious layer and the treatment fluid dispensing layer overlying one another, having identical shapes, said shapes being selected from the group consisting of strips, patches, and disks.
